(19) United States
(12) Patent Application Publication
(10) Pub. No.: US 2023/0175393 A1
(43) Pub. Date: Jun. 8, 2023

(54) ESTIMATING COMPOSITION OF DRILLING FLUID IN A WELLBORE USING DIRECT AND INDIRECT MEASUREMENTS

(71) Applicant: Halliburton Energy Services, Inc., Houston, TX (US)

(72) Inventors: Sandeep Dileep Kulkarni, Houston, TX (US); Lalit Narayan Mahajan, Fremont, CA (US); Dale E. Jamison, Humble, TX (US)

(21) Appl. No.: 17/546,009

(22) Filed: Dec. 8, 2021

(51) Int. Cl. E21B 49/00; E21B 49/08; G01N 33/28; G06Q 50/02

(52) U.S. Cl. CPC E21B 49/003; E21B 49/0875; G01N 33/2823; G06Q 50/02; E21B 49/005

(57) ABSTRACT

A system can receive a combination of measurements of a drilling fluid from a wellbore during a wellbore drilling operation. The system can determine, using the combination of measurements, at least one component of the drilling fluid of the drilling fluid estimate. The system can determine, using the at least one component of the drilling fluid, a composition of the drilling fluid. The system can output the composition of the drilling fluid for use in controlling the wellbore drilling operation.

### TECHNICAL FIELD

The present disclosure relates generally to wellbore drilling operations and, more particularly (although not necessarily exclusively), to using direct and indirect measurements to estimate a composition of drilling fluid for a wellbore drilling operation.

### BACKGROUND

A drilling fluid can be added to a wellbore to facilitate a wellbore drilling operation by suspending cuttings, controlling a pressure in the wellbore, stabilizing exposed portions of rock in the wellbore, or cooling and lubricating the drill bit. The drilling fluid can include water, oil, a mud, a synthetic fluid, or a combination of multiple fluids. A composition of the drilling fluid can be measured using a rheological test such as a retort test, which can provide information about the relative proportions of oil, water, and drilling solids in the drilling fluid. But such a test involves multiple steps and can involve a significant amount of time, delaying knowledge about the composition of the drilling fluid.

### BRIEF DESCRIPTION OF THE DRAWINGS

FIG. 1 is a schematic diagram of a drilling system for drilling a wellbore using drilling fluid according to one example of the present disclosure.

FIG. 2 is a flowchart of a process for determining a composition of a drilling fluid for a drilling operation according to one example of the present disclosure.

FIG. 3 is a block diagram of a computing device that can determine a composition of a drilling fluid according to one example of the present disclosure.

### DETAILED DESCRIPTION

Certain aspects and examples of the present disclosure relate to using a combination of direct and indirect measurements to estimate a composition of a drilling fluid. The composition of the drilling fluid may be characterized in part by values for an oil water ratio and an average specific gravity, which can be estimated by the combination of direct and indirect measurements. The oil water ratio and the average specific gravity of solids can be used to determine information about conditions in the wellbore, and can be used to adjust drilling parameters. The combination of direct and indirect measurements may be retrieved from a sensor component that can be deployed downhole in the wellbore. A direct measurement may be a measurement of a desired quantity, such as a fluid density measurement that can yield a value of density for a drilling fluid. An indirect measurement may be a measurement of a quantity that can be related to the desired quantity. An example of an indirect measurement can include a thermal conductivity measurement that can be used for estimating at least one component of the drilling fluid of the drilling fluid.

In some cases, direct measurements of quantities associated with the composition of the drilling fluid, such as oil water ratio and average specific gravity, can be difficult or time-consuming. Oil water ratio and average specific gravity can be determined using a rheological test such as a retort test. But such a test involves multiple steps and can involve a significant amount of time, delaying knowledge about the composition of the drilling fluid. This delay can prevent drilling events such as sag, lost circulation, kick, influx, and cuttings accumulation from being detected quickly. The delay can also prevent drilling parameters from being adjusted substantially contemporaneously with determining the oil water ratio and average specific gravity.

In some examples of the present disclosure, the combination of direct and indirect measurements of the drilling fluid may be easy to automate. The combination of direct and indirect measurements can be used to determine or estimate the composition of the drilling fluid in real time, such as by determining or estimating the composition substantially contemporaneously with acquiring the combination of direct and indirect measurements. Real-time estimation of the composition of the drilling fluid may allow a drilling event, such as sag, lost circulation, kick, influx, or cuttings accumulation, to be detected. In some examples, estimating the composition may also allow for an operational parameter to be monitored and adjusted using the output of estimating the composition. Estimating the composition of the drilling fluid substantially contemporaneously to monitoring and adjusting operational parameters may allow a subterranean rock formation around the wellbore to be better stabilized, and may allow a stability of the formation to be predicted ahead of a bit. In some examples, the estimate of the composition may be used to adjust wellbore hydraulics or fluid component dosing.

In some examples of the present disclosure, a combination of direct and indirect fluid measurements can be used to estimate the composition of the drilling fluid. The combination of direct and indirect measurements can be received from a sensor component positioned in the wellbore. The combination of direct and indirect measurements may include measurements of density, thermal conductivity, water-retort, and salinity. The direct and indirect measurements may be gathered at different locations in the wellbore. The direct and indirect measurements may be automated and may be used in combination with electrical sensors. The electrical sensors may be optical fiber sensors or wireless chip sensors. The direct and indirect measurements may be gathered at or adjusted for a fixed temperature and pressure. The memory may further include instructions to use the combination of measurements to determine at least one component of the fluid. In one example, determining a component of the fluid can involve determining a volume fraction of the drilling fluid. The combination of direct and indirect measurements can be used to estimate, using the component(s) of the drilling fluid, the composition of the drilling fluid. The composition of the drilling fluid can be characterized in part by an oil water ratio and an average specific gravity of solids that may be present in the drilling fluid. The memory may further include material libraries, which may include density values for each component of the drilling fluid, thermal conductivity values of each component of the drilling fluid, or both. The material libraries can be used to determine the component(s) of the drilling fluid of the drilling fluid.

An average specific gravity of a solid may be a ratio of the average density of the solids to the density of water at twenty (20) degrees Celsius. High gravity solids may be solids with a relatively high density that can be suspended in a wellbore, such as barite or hematite. On the other hand, low gravity solids may be solids with a relatively low density that can be suspended in a wellbore, such as drill cuttings and bentonite clay.

Illustrative examples are given to introduce the reader to the general subject matter discussed herein and are not intended to limit the scope of the disclosed concepts. The following sections describe various additional features and examples with reference to the drawings in which like numerals indicate like elements, and directional descriptions are used to describe the illustrative aspects, but, like the illustrative aspects, should not be used to limit the present disclosure.

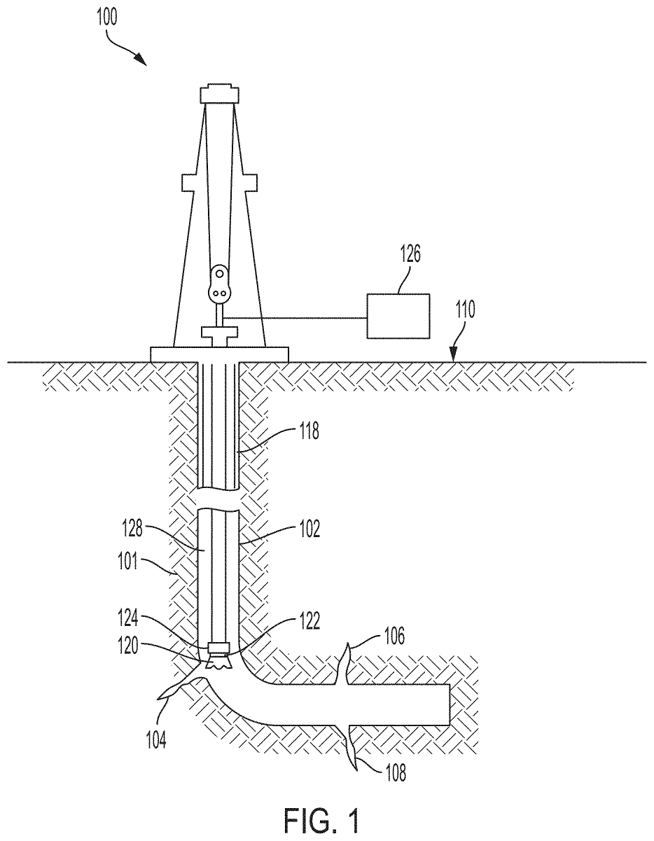

FIG. 1 is a schematic diagram of a drilling system for drilling a wellbore using drilling fluid according to one example of the present disclosure. In this example, drilling rig 100 is depicted for a well, such as an oil or gas well, for extracting fluids from a subterranean formation 101. The drilling rig 100 may be used to create a wellbore 102 from a surface 110 of the subterranean formation 101. The drilling rig 100 may include a drilling subassembly 118, and a drill bit 120. The downhole tool 118 can be any tool used to gather information about the wellbore 102. For example, the downhole tool 118 can be a tool used for measuring-while-drilling or logging-while-drilling operations. The downhole tool 118 can include a sensor component 122 for collecting measurements from the wellbore 102. The downhole tool 118 can also include a transmitter 124 for transmitting data from the sensor component 122 to the surface 110. A bottom hole assembly 134 can include the downhole tool 118 and the drill bit 120 for drilling the wellbore 102.

The wellbore 102 is shown as being drilled from the surface 110 and through the subterranean formation 101. As the wellbore 102 is drilled, drilling fluid can be pumped through the drill bit 120 and into the wellbore 102 to enhance drilling operations. The drilling fluid can enhance the drilling operation by suspending cuttings, controlling a pressure in the wellbore, stabilizing exposed portions of rock in the wellbore, or cooling and lubricating the drill bit. As the drilling fluid enters into the wellbore, the drilling fluid circulates back toward the surface 110 through a wellbore annulus 128, which is an area between a drill string 130 and a wall 132 of the wellbore 102. Also included in the schematic diagram is a computing device 126. The computing device 126 can be communicatively coupled to the downhole tool 118 and receive real-time information about the drilling operation. The computing device 126 can determine parameters for the drilling operation and cause adjustments to parameters of the drilling operation.

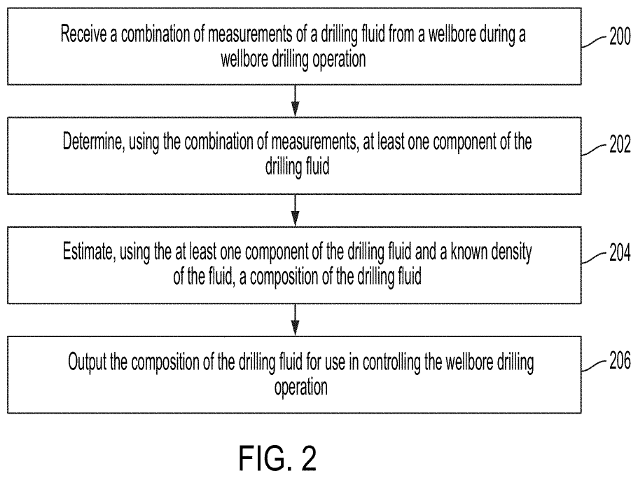

FIG. 2 is a flowchart of a process for determining a composition of a drilling fluid for a drilling operation according to one example of the present disclosure.

The composition of the drilling fluid may be characterized in part by an oil water ratio and an average specific gravity of solids in a wellbore.

In block 200, a computing device receives a combination of measurements of the drilling fluid from the wellbore during a wellbore drilling operation. The combination of direct and indirect measurements may be a combination of direct and indirect measurements, which can be received from a sensor component positioned in the wellbore. The sensor component may be a sensor plug that can be coupled to a surface control unit. The combination of direct and indirect measurements may include measurements of density, thermal conductivity, water-retort, and salinity. The direct and indirect measurements may be automated and may be used in combination with data from electrical sensors. The direct and indirect measurements may be gathered at or adjusted for a fixed temperature and pressure. The computing device may use material libraries, which may include density values for each component of the drilling fluid, thermal conductivity values of each component of the drilling fluid, or both. The material libraries can be used to determine the at least one component of the drilling fluid of the drilling fluid.

In block 202, the computing device determines, using the combination of direct and indirect measurements, the at least one component of the drilling fluid. For example, a volume fraction of the drilling fluid can be determined. The volume fraction may include a volume of oil, a volume of brine, a volume of low gravity solids, and a volume of high gravity solids. High gravity solids may be solids with a relatively high density that can be suspended in a wellbore, such as barite or hematite. Low gravity solids may be solids with a relatively low density that can be suspended in a wellbore, such as drill cuttings and bentonite clay. To determine the at least one component of the drilling fluid, the computing device can determine a volume balance of the drilling fluid, where a sum of every element in the volume balance can be equal to one. In other words, each element in the volume balance can be expressed as a fraction of one and can represent a percentage of a full volume of the drilling fluid when multiplied by 100. The computing device can also determine a mass balance of the drilling fluid with a sum of products of density components and volume components for use in estimating the composition of the drilling fluid.

Like the volume balance, the mass balance also can allow each element in the mass balance to be expressed as a fraction of one and can allow every element in the mass balance to represent a percentage of a full mass of the drilling fluid when multiplied by 100. The computing device can also determine a thermal conductivity of the drilling fluid. The thermal conductivity of the drilling fluid can be expressed as a function of fluid components, where the function of fluid components can include: a linear function, a power function, an exponential function, a polynomial function, or any combination thereof. Using the volume balance and the mass balance and the thermal conductivity, at least one component of the drilling fluid of the components of drilling fluid can be determined.

In block 204, the computing device estimates, using the at least one component of the drilling fluid and a known density of the drilling fluid, a composition of the drilling fluid. The composition can be estimated or determined by calculating quantities such as an oil water ratio and an average specific gravity of solids suspended in the drilling fluid. The oil water ratio may be calculated by dividing the volume of oil by a sum of the volume of oil and the volume of water and multiplying the result by 100. The average specific gravity of solids suspended in the drilling fluid may be a ratio of the average density of the solids suspended in the drilling fluid to the density of water at twenty (20) degrees Celsius. Examples of solids suspended in the drilling fluid can include high gravity solids and low gravity solids. High gravity solids may be solids with a relatively high density that can be suspended in a wellbore, such as barite or hematite. Low gravity solids may be solids with a relatively low density that can be suspended in a wellbore, such as drill cuttings and bentonite clay. The average specific gravity (ASG) of solids in the drilling fluid can be calculated with Equation 1, using a volume of low gravity solids V_{lgs}, a density of low gravity solids ρ_{lgs}, a volume of high gravity solids V_{hgs}, and a density of high gravity solids ρ_{hgs}.

$$ASG = \frac{{V}_{lgs} * {ρ}_{lgs} + {V}_{hgs} * {ρ}_{hgs}}{{V}_{lgs} + {V}_{hgs}} \qquad ( 1 )$$

In block 206, a computing device outputs the composition of the drilling fluid for use in controlling a wellbore drilling operation. The output can be used to adjust drilling parameters or to stabilize a rock formation ahead-of-bit. Drilling parameters that may be adjusted can include, but are not limited to: centrifuge speed, weight-on-bit, rate of penetration, and rotations per minute. Adjusting these parameters based on estimates of the composition of the drilling fluid may result in more stable wellbore conditions.

The output can also be used to detect drilling events. In some examples, the output can be used to detect sag, where the drilling fluid may be heavier towards a bottom of the wellbore. The output can also be used to detect a cuttings accumulation, where rock from the wellbore may fall into the drilling fluid. The output can also be used to detect a kick, where formation fluids may flow into the wellbore due to a pressure differential.

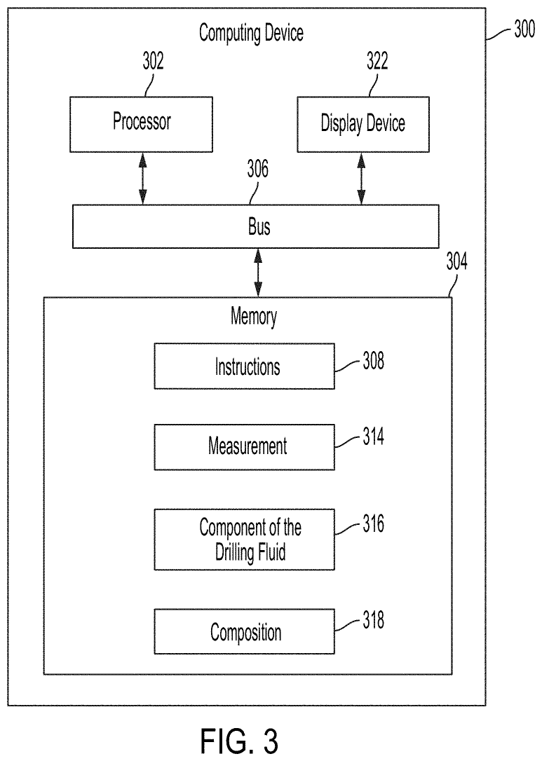

FIG. 3 is a block diagram of a computing device 300 that can determine a composition of a drilling fluid according to one example of the present disclosure. The computing device 300 can include a processor 302, a bus 306, and a memory 304. In some examples, the components shown in FIG. 3 can be integrated into a single structure. For example, the components can be within a single housing with a single processing device. In other examples, the components shown in FIG. 3 can be distributed (e.g., in separate housings) and in electrical communication with each other using various processors. It is also possible for the components to be distributed in a cloud computing system or grid computing system.

The processor 302 can execute one or more operations for determining an operating window. The processor 302 can execute instructions 308 stored in the memory 304 to perform the operations. The processor 302 can include one processing device or multiple processing devices. Non-limiting examples of the processor 302 include a field-programmable gate array (“FPGA”), an application-specific integrated circuit (“ASIC”), a processor, a microprocessor, etc.

The processor 302 is communicatively coupled to the memory 304 via the bus 306. The memory 304 may include any type of memory device that retains stored information when powered off. Non-limiting examples of the memory 304 include electrically erasable and programmable read-only memory (“EEPROM”), flash memory, or any other type of non-volatile memory. In some examples, at least some of the memory 304 can include a non-transitory medium from which the processor 302 can read the instructions 308. A computer-readable medium can include electronic, optical, magnetic, or other storage devices capable of providing the processor 302 with computer-readable instructions or other program code. Non-limiting examples of a computer-readable medium include (but are not limited to) magnetic disk(s), memory chip(s), read-only memory (ROM), random-access memory (“RAM”), an ASIC, a configured processing device, optical storage, or any other medium from which a computer processing device can read instructions. The instructions can include processing device-specific instructions generated by a compiler or an interpreter from code written in any suitable computer-programming language, including, for example, C, C++, C#, etc. The instructions may include a drilling fluid composition analysis engine 308. The memory 304 may store a measurement 314. The measurement 314 may be a direct or indirect measurement, and can be used by the drilling fluid composition analysis engine 308 to determine a at least one component of the drilling fluid 316. The at least one component of the drilling fluid may be determined by, using the measurement 314 and the drilling fluid composition analysis engine 308, calculating a mass balance, a volume balance, and a thermal conductivity. The at least one component of the drilling fluid 316 can be used by the drilling fluid composition analysis engine 308 to determine a composition 318 of the drilling fluid. The composition 318 can be estimated or determined by calculating an oil water ratio and an average specific gravity of the drilling fluid.

The oil water ratio may be calculated by dividing the volume of oil by a sum of the volume of oil and the volume of water and multiplying the result by 100. The average specific gravity of solids suspended in the drilling fluid may be a ratio of the average density of the solids to the density of water at twenty (20) degrees Celsius. Examples of solids suspended in the drilling fluid can include high gravity solids and low gravity solids. High gravity solids may be solids with a relatively high density that can be suspended in a wellbore, such as barite or hematite. Low gravity solids may be solids with a relatively low density that can be suspended in a wellbore, such as drill cuttings and bentonite clay. The average specific gravity (ASG) of solids in the drilling fluid can be calculated with Equation 1, using a volume of low gravity solids V_{lgs}, a density of low gravity solids ρ_{lgs}, a volume of high gravity solids V_{hgs}, and a density of high gravity solids ρ_{hgs}.

In some aspects, system, method, and non-transitory computer-readable medium for estimating a composition of a drilling fluid are provided according to one or more of the following examples:

Example 1 is a system comprising a processor and a memory that includes instructions executable by the processor for causing the processor to receive a combination of measurements of a drilling fluid from a wellbore during a wellbore drilling operation, determine, using the combination of measurements, at least one component of the drilling fluid, estimate, using the at least one component of the drilling fluid, a composition of the drilling fluid, and output the composition of the drilling fluid for use in controlling the wellbore drilling operation.

Example 2 is the system of example 1, wherein the instructions are executable by the processor for causing the processor to estimate, using the at least one component of the drilling fluid, the composition of the drilling fluid by: determining an oil water ratio of the drilling fluid and determining an average specific gravity of the drilling fluid.

Example 3 is the system of example 1, further comprising instructions executable by the processor for causing the processor to: output a first command to adjust wellbore hydraulics, output a second command to adjust an operational parameter, and output a third command to adjust a fluid component dosing.

Example 4 is the system of example 1, wherein the combination of measurements comprises at least one of: a density measurement, a thermal conductivity measurement, a water-retort measurement, or a salinity measurement, wherein the combination of measurements is acquirable at multiple locations at the wellbore, and wherein the combination of measurements is gatherable at, or adjustable for, a temperature and a pressure.

Example 5 is the system of example 1, wherein the instructions are executable by the processor for causing the processor to estimate, using the at least one component of the drilling fluid, the composition of the drilling fluid by: determining a volume balance of the drilling fluid with a sum of volume components, determining a mass balance of the drilling fluid with a sum of products of density components and volume components, determining a thermal conductivity of the drilling fluid, wherein the thermal conductivity of the drilling fluid is expressed as a function of fluid components, wherein the function of fluid components includes at least one of: a linear function, a power function, an exponential function, a polynomial function, or any combination thereof, and determining, using the volume balance and the mass balance and the thermal conductivity, an at least one component of the drilling fluid of the drilling fluid.

Example 6 is the system of example 1, further comprising a second combination of measurements retrievable from an electrical sensor.

Example 7 is the system of example 1, wherein the instructions are executable by the processor for causing the processor to estimate, using the at least one component of the drilling fluid, the composition of the drilling fluid by determining an oil water ratio of the drilling fluid by dividing the volume of oil by a sum of the volume of oil and the volume of water and multiplying the result by 100, and determining an average specific gravity by taking a sum of a product of a volume of low gravity solids and a density of low gravity solids with a product of a volume of high gravity solids and a density of high gravity solids and dividing the sum by a second sum of the volume of low gravity solids and the volume of high gravity solids.

Example 8 is a method comprising: receiving, by a processor, a combination of measurements of a drilling fluid from a wellbore during a wellbore drilling operation, determining, by the processor and using the combination of measurements, a at least one component of the drilling fluid of the drilling fluid, estimating, by the processor and using the at least one component of the drilling fluid, a composition of the drilling fluid, and outputting, by the processor, the composition of the drilling fluid.

Example 9 is the method of example 8, further comprising: determining, by the processor, an oil water ratio of the fluid for estimating the composition of the drilling fluid, and determining, by the processor, an average specific gravity of the drilling fluid for estimating the composition of the drilling fluid.

Example 10 is the method of example 8, further comprising: outputting a first command, by the processor to adjust wellbore hydraulics, outputting a second command, by the processor, to adjust an operational parameter, and outputting a third command, by the processor to adjust a fluid component dosing.

Example 11 is the method of example 8, wherein the combination of measurements comprises at least one of: a density measurement, a thermal conductivity measurement, a water-retort measurement, and a salinity measurement, wherein the combination of measurements is acquirable at multiple locations at the wellbore, and wherein the combination of measurements is gatherable at, or adjustable for, a temperature and a pressure.

Example 12 is the method of example 8, further comprising: determining, by the processor, a volume balance of the drilling fluid, determining, by the processor, a mass balance of the drilling fluid by summing products of density components and volume components, determining, by the processor, a thermal conductivity of the drilling fluid, wherein the thermal conductivity of the drilling fluid is expressed as a function of fluid components, wherein the function of fluid components includes at least one of: a linear function, a power function, an exponential function, a polynomial function, or any combination thereof; and determining, by the processor, and using the volume balance and the mass balance and the thermal conductivity, a at least one component of the drilling fluid of the drilling fluid.

Example 13 is the method of example 8, further comprising retrieving a second combination of measurements from an electrical sensor.

Example 14 is the method of example 8, further comprising: determining, using the at least one component of the drilling fluid, an oil water ratio of the drilling fluid by dividing the volume of oil by a sum of the volume of oil and the volume of water and multiplying the result by 100; and determining, using the at least one component of the drilling fluid, an average specific gravity of the drilling fluid by taking a sum of a product of a volume of low gravity solids and a density of low gravity solids with a product of a volume of high gravity solids and a density of high gravity solids and dividing the sum by a second sum of the volume of low gravity solids and the volume of high gravity solids.

Example 15 is a non-transitory computer-readable medium comprising instructions that are executable by a processor for causing the processor to perform operations comprising: receiving a combination of measurements of a drilling fluid from a wellbore during a wellbore drilling operation, determining, using the combination of measurements, a at least one component of the drilling fluid of the drilling fluid, estimating, using the at least one component of the drilling fluid, a composition of the drilling fluid, and outputting the composition of the drilling fluid for use in controlling the wellbore drilling operation.

Example 16 is the non-transitory computer-readable medium of example 15, wherein the operation of estimating, using the combination of measurements, the composition further comprises: determining an oil water ratio of the fluid and determining an average specific gravity of the drilling fluid.

Example 17 is the non-transitory computer-readable medium of example 15, further comprising instructions that are executable by a processor for causing the processor to perform operations comprising: outputting a first command, by the processor to adjust wellbore hydraulics, outputting a second command, by the processor, to adjust an operational parameter, and outputting a third command, by the processor to adjust a fluid component dosing.

Example 18 is the non-transitory computer-readable medium of example 15, wherein the combination of measurements comprises at least one of: a density measurement, a thermal conductivity measurement, a water-retort measurement, and a salinity measurement and wherein the combination of measurements is gatherable at or adjustable for a temperature and a pressure.

Example 19 is the non-transitory computer-readable medium of example 15, wherein the instructions are executable by the processor for causing the processor to estimate, using the at least one component of the drilling fluid, the composition of the drilling fluid by: determining an oil water ratio of the drilling fluid by dividing the volume of oil by a sum of the volume of oil and the volume of water and multiplying the result by 100, and determining an average specific gravity of the drilling fluid by taking a sum of a product of a volume of low gravity solids and a density of low gravity solids with a product of a volume of high gravity solids and a density of high gravity solids and dividing the sum by a second sum of the volume of low gravity solids and the volume of high gravity solids.

Example 20 is the non-transitory computer-readable medium of example 15, wherein the instructions are further executable by the processor for causing the processor to estimate the composition of the drilling fluid by retrieving a second combination of measurements from an electrical sensor.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

1. A system comprising:
a processor; and
a memory that includes instructions executable by the processor for causing the processor to:
receive a combination of measurements of a drilling fluid from a wellbore during a wellbore drilling operation;
determine, using the combination of measurements, at least one component of the drilling fluid;
estimate, using the at least one component of the drilling fluid, a composition of the drilling fluid by:
determining an average specific gravity of solids in the drilling fluid by taking a sum of a product of a volume of low gravity solids and a density of low gravity solids with a product of a volume of high gravity solids and a density of high gravity solids and dividing the sum by a product of a density of water and a second sum of the volume of low gravity solids and the volume of high gravity solids, wherein the low gravity solids consist of at least one material selected from the group consisting of: (i) bentonite clay and (ii) drill cuttings, and wherein the high gravity solids have a higher specific gravity than the low gravity solids; and
output the composition of the drilling fluid for use in controlling the wellbore drilling operation.

2. The system of claim 1, wherein the instructions are executable by the processor for causing the processor to further estimate, using the at least one component of the drilling fluid, the composition of the drilling fluid by:
determining an oil water ratio of the drilling fluid.

3. The system of claim 1, further comprising instructions executable by the processor for causing the processor to:
output a first command to adjust wellbore hydraulics based on the composition of the drilling fluid;
output a second command to adjust an operational parameter based on the composition of the drilling fluid; and
output a third command to adjust a fluid component dosing of the at least one fluid component based on the composition of the drilling fluid.

4. The system of claim 1, wherein the combination of measurements comprises at least one of: a density measurement, a thermal conductivity measurement, a water-retort measurement, or a salinity measurement,
wherein the combination of measurements is acquirable at multiple locations at the wellbore, and
wherein the combination of measurements is gatherable at, or adjustable for, a temperature and a pressure.

5. The system of claim 1, wherein the instructions are executable by the processor for causing the processor to estimate, using the at least one component of the drilling fluid, the composition of the drilling fluid by:
determining a volume balance of the drilling fluid with a sum of volume components;
determining a mass balance of the drilling fluid with a sum of products of density components and volume components;
determining a thermal conductivity of the drilling fluid, wherein the thermal conductivity of the drilling fluid is expressed as a combination of fluid components of the drilling fluid; and
determining, using the volume balance. the mass balance and the thermal conductivity, at least one component of the drilling fluid.

6. The system of claim 1, wherein the instructions are further executable by the processor for causing the processor to estimate, using the at least one component of the drilling fluid, the composition of the drilling fluid by:
retrieving a second combination of measurements from an electrical sensor that is positioned in the wellbore, wherein the second combination of measurements comprises direct measurements that are usable to determine the at least one component of the drilling fluid.

7. (canceled)

8. A computer-implemented method comprising:
receiving, by a computer processor, a combination of measurements of a drilling fluid from a sensor component that is positioned in a wellbore during a wellbore drilling operation;
determining, by the computer processor and using the combination of measurements, at least one component of the drilling fluid;
estimating, by the computer processor and using the at least one component of the drilling fluid, a composition of the drilling fluid by:
determining an average specific gravity of solids in the drilling fluid by taking a sum of a product of a volume of low gravity solids and a density of low gravity solids with a product of a volume of high gravity solids and a density of high gravity solids and dividing the sum by a product of a density of water and a second sum of the volume of low gravity solids and the volume of high gravity solids, wherein the low gravity solids consist of at least one material selected from the group consisting of: (i) bentonite clay and (ii) drill cuttings, and wherein the high gravity solids have a higher specific gravity than the low gravity solids; and
outputting, by the computer processor, the composition of the drilling fluid for use in controlling the wellbore drilling operation.

9. The method of claim 8, further comprising:
determining, by the computer processor, an oil water ratio of the fluid for estimating the composition of the drilling fluid.

10. The method of claim 8, further comprising:
outputting a first command, by the computer processor to adjust wellbore hydraulics based on the composition of the drilling fluid;
outputting a second command, by the computer processor, to adjust an operational parameter based on the composition of the drilling fluid; and
outputting a third command, by the computer processor to adjust a fluid component dosing of the at least one fluid component based on the composition of the drilling fluid.

11. The method of claim 8, wherein the combination of measurements comprises at least one of: a density measurement, a thermal conductivity measurement, a water-retort measurement, and a salinity measurement,
wherein the combination of measurements is acquirable at multiple locations at the wellbore, and
wherein the combination of measurements is gatherable at, or adjustable for, a temperature and a pressure.

12. The method of claim 8, further comprising:
determining, by the computer processor, a volume balance of the drilling fluid;
determining, by the computer processor, a mass balance of the drilling fluid by summing products of density components and volume components;
determining, by the computer processor, a thermal conductivity of the drilling fluid, wherein the thermal conductivity of the drilling fluid is expressed as a combination of fluid components of the drilling fluid; and
determining, by the computer processor, and using the volume balance, the mass balance, and the thermal conductivity, at least one component of the drilling fluid of the drilling fluid.

13. The method of claim 8, further comprising retrieving a second combination of measurements from an electrical sensor that is positioned in the wellbore, wherein the second combination of measurements comprises direct measurements that are usable to determine the at least one component of the drilling fluid.

14. (canceled)

15. A non-transitory computer-readable medium comprising instructions that are executable by a processor for causing the processor to perform operations comprising:
receiving a combination of measurements of a drilling fluid from a wellbore during a wellbore drilling operation;
determining, using the combination of measurements, at least one component of the drilling fluid;
estimating, using the at least one component of the drilling fluid, a composition of the drilling fluid by:
determining an average specific gravity of solids in the drilling fluid by taking a sum of a product of a volume of low gravity solids and a density of low gravity solids with a product of a volume of high gravity solids and a density of high gravity solids and dividing the sum by a product of a density of water and a second sum of the volume of low gravity solids and the volume of high gravity solids, wherein the low gravity solids consist of at least one material selected from the group consisting of: (i) bentonite clay and (ii) drill cuttings, and wherein the high gravity solids have a higher specific gravity than the low gravity solids; and
outputting the composition of the drilling fluid for use in controlling the wellbore drilling operation.

16. The non-transitory computer-readable medium of claim 15, wherein the operation of estimating, using the combination of measurements, the composition further comprises:
determining an oil water ratio of the fluid.

17. The non-transitory computer-readable medium of claim 15, further comprising instructions that are executable by a processor for causing the processor to perform operations comprising:
outputting a first command, by the processor to adjust wellbore hydraulics based on the composition of the drilling fluid;
outputting a second command, by the processor, to adjust an operational parameter based on the composition of the drilling fluid; and
outputting a third command, by the processor to adjust a fluid component dosing of the at least one fluid component based on the composition of the drilling fluid.

18. The non-transitory computer-readable medium of claim 15, wherein the combination of measurements comprises at least one of: a density measurement, a thermal conductivity measurement, a water-retort measurement, and a salinity measurement and wherein the combination of measurements is gatherable at or adjustable for a temperature and a pressure.

19. (canceled)

20. The non-transitory computer-readable medium of claim 15, wherein the instructions are further executable by the processor for causing the processor to estimate the composition of the drilling fluid by: retrieving a second combination of measurements from an electrical sensor that is positioned in the wellbore, wherein the second combination of measurements comprise direct measurements that are usable to determine the at least one component of the drilling fluid.

21. The system of claim 1, wherein the instructions are executable by the processor for causing the processor to determine the at least one component of the drilling fluid by:
retrieving, from at least one material library, at least one of: (i) a density value for the component of the drilling fluid, (ii) or a thermal conductivity value for the component of the drilling fluid.

22. The method of claim 8, wherein determining the at least one component of the drilling fluid further comprises:
retrieving, by the processor and from at least one material library, at least one of: (i) a density value for the component of the drilling fluid, (ii) or a thermal conductivity value for the component of the drilling fluid.

23. The non-transitory computer-readable medium of claim 15, further comprising instructions that are executable by a processor for causing the processor to determine the at least one component of the drilling fluid by:
retrieving, from at least one material library, at least one of: (i) a density value for the component of the drilling fluid, (ii) or a thermal conductivity value for the component of the drilling fluid.
